(19) United States
(12) Patent Application Publication
(10) Pub. No.: US 2022/0106371 A1
(43) Pub. Date: Apr. 7, 2022

(54) MODIFIED RECOMBINANT HUMAN NERVE GROWTH FACTOR AND METHOD FOR PREPARING THE SAME

(71) Applicant: Xintrum Pharmaceuticals, Ltd., Nanjing (CN)

(72) Inventors: Hai Chen, Nanjing (CN); Gaoyong Liao, Nanjing (CN); Yi Zhang, Nanjing (CN)

(21) Appl. No.: 17/555,375

(22) Filed: Dec. 18, 2021

Related U.S. Application Data
(63) Continuation of application No. PCT/CN2021/102050, filed on Jun. 24, 2021.

(30) Foreign Application Priority Data
Jun. 28, 2020 (CN) 202010595084.3

(51) Int. Cl. C07K 14/48

(52) U.S. Cl. CPC C07K 14/48; A61K 38/00

(57) ABSTRACT

A modified recombinant human nerve growth factor (modified rhNGF) is obtained from the reaction between a polymer of formula A and an rhNGF. The polymer of formula A is an N-disubstituted amino acetamino aldehyde derivative. Experimental results have shown that the modified rhNGF has a higher in vivo plasma concentration and a longer in vivo half-life than when the rhNGF is not modified or is modified by monomethoxy polyglycol, and that the modified rhNGF preserves the original activity of the unmodified rhNGF. Moreover, the method of preparing the modified rhNGF is low-cost, and the modified products are highly consistent.

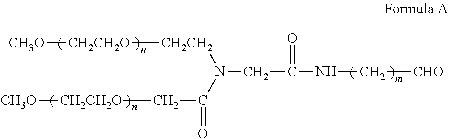

### TECHNICAL FIELD

The present invention relates to the field of biopharmaceuticals and more particularly to a modified recombinant human nerve growth factor (modified rhNGF) and a method for preparing the same.

### DESCRIPTION OF RELATED ART

Nerve growth factor (NGF) is a nerve cell growth regulating factor that has important biological functions. NGF plays an important role in regulating the development, differentiation, growth, regeneration, and expression of functional properties of both the central and the peripheral nervous systems. NGF can promote the maturation of sympathetic neurons and sensory neurons and sustain the normal functions of mature sympathetic neurons.

NGF is a protein molecule and therefore has a relatively short half-life after entering the human body. In order for the NGF in a patient's body to stay active, daily administration of NGF is required, but patient compliance is relatively low. It is hence imperative to protect the molecule from degradation, and thereby lower the removal rate and extend the half-life, of such a protein-based drug so as to reduce the frequency and dose level of drug administration.

Protein modification, such as by binding a biologically inert, safe, and non-toxic polymer covalently to a natural protein, is often effective in improving the clinical properties of a drug based on the protein, e.g., in increasing the plasma half-life, reducing the immunogenicity, and enhancing the efficacy and safety of the drug.

However, as a protein molecule generally has more than one binding site to the polymer modifier, some protein molecules may bind to multiple modifiers as well as a single modifier during the protein modification process, and compared with a singly modified product, a multiply modified product not only lacks batch-to-batch consistency, presents difficulty in quality control, constitutes a wasteful use of material, and leads to high economic cost, but also tends to suffer a great loss in original bioactivity.

Accordingly, it is necessary to optimize the modification conditions so that the modification reaction involves more single modification than multiple modifications, the goal being to render the modified products more consistent and preserve the original bioactivity of the corresponding natural protein.

### BRIEF SUMMARY OF THE INVENTION

One objective of the present invention is to modify a recombinant human nerve growth factor (hereinafter also referred to as rhNGF) and thereby increase the in vivo stability, and extend the half-life, of the rhNGF. The modification conditions will also be optimized so that the modified product maintains its original bioactivity.

According to the present invention, a polymer of formula A and an N-terminal α-amino group of an rhNGF are allowed to bind covalently to each other to produce a modified rhNGF (formula B):

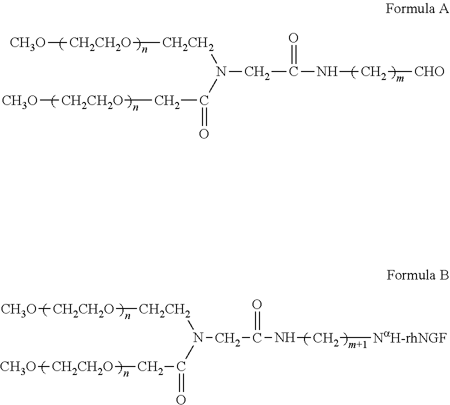

where m is 1 or 2.

The polymer of formula A is an N-disubstituted amino acetamino aldehyde derivative.

The polymer of formula A has a weight-average molecular weight of 10 KD-40 kD, preferably 20 kD or 40 kD.

The rhNGF is prepared by a recombinant DNA technique, or more specifically by forming a dimer structure out of two identical single chains whose amino acid sequences are SEQ ID NO. 1 or SEQ ID NO. 2. That is to say, the rhNGF is composed of two single-chain of amino acids of the sequence of SEQ ID NO. 1 or two single-chain of amino acids of the sequence of SEQ ID NO. 2.

Research on the method of preparing the modified rhNGF (formula B)

The major technical difficulty in binding the polymer of formula A to the rhNGF is to control the binding reaction and bring about more single modification than multiple modifications so as to obtain singly modified products that are consistent and that maintain the original bioactivity of the rhNGF protein. To overcome this difficulty, the following research was conducted for the present invention:

The various reaction conditions involved in modifying the rhNGF with the polymer of formula A were thoroughly analyzed by the response surface methodology by using Design of Experiment (DOE) peogram, and the optimal conditions obtained include:

a molar ratio of the polymer of formula A to the rhNGF that enables highly efficient modification, a preferred range of pH values, and a suitable reaction solvent, reaction temperature, and reaction time.

The present invention provides a method for preparing the modified rhNGF (formula B): the method includes reacting the rhNGF with the polymer of formula A in the presence of sodium cyanoborohydride serving as a reducing agent, in order to obtain the modified rhNGF (formula B).

The molar ratio of the polymer of formula A to the rhNGF is 1-2:1.

The final concentration of the reducing agent, i.e., sodium cyanoborohydride, is 20 mM.

The reaction solvent is an acetic acid/sodium acetate buffer solution, and a preferred pH value is 5.0-5.8.

The reaction temperature is 5±3° C. or 25±2° C.

The reaction time is 2 h-24 h.

The present invention has the following advantageous effects:

1. Extending the in vivo half-life of the rhNGF

A pharmacokinetic research on the modified rhNGF was conducted in the bodies of rats. The research results show that the modified rhNGF provided by the present invention had a higher in vivo plasma concentration and a longer in vivo half-life than when the rhNGF was not modified or was modified by monomethoxy polyglycol (see embodiment 5).

2. Preserving the original bioactivity of the unmodified rhNGF

The bioactivity of the modified rhNGF was determined by a TF-1 cell/MTS colorimetric assay. The assay results show that the bioactivity in promoting TF-1 cell proliferation was preserved (see embodiment 6). In other words, the original activity of the unmodified rhNGF was preserved.

3. Low-cost preparation method and highly consistent modified products

The method provided by the present invention for preparing the modified rhNGF features high reaction activity, uses relatively small amounts of materials, and produces highly consistent modified products, the percentage of singly modified products being greater than 83% (see embodiments 1, 2, 3, and 4).

### BRIEF DESCRIPTION OF THE SEVERAL VIEWS OF THE DRAWINGS

FIG. 1 shows SDS-PAGE test results of the products of reactions in which an rhNGF is modified by different formula-A polymers separately, with M being molecular-weight-standard protein markers, and lanes 1-5 corresponding respectively to the rhNGF, a polymer of formula A-20K, modified product LAP2-20K, a polymer of formula A-40K, and modified product LAP2-40K;

FIG. 2 shows a DOE test result, or more specifically a single-modification percentage contour map, corresponding to a reaction in which an rhNGF is modified by a polymer of formula A-20K;

FIG. 3 shows a DOE test result, or more specifically a multiple-modification percentage contour map, corresponding to a reaction in which an rhNGF is modified by a polymer of formula A-20K;

FIG. 4 shows a DOE test result, or more specifically a contour line overlay plot, corresponding to a reaction in which an rhNGF is modified by a polymer of formula A-20K;

FIG. 5 shows a DOE test result, or more specifically a single-modification percentage contour map, corresponding to a reaction in which an rhNGF is modified by a polymer of formula A-40K;

FIG. 6 shows a DOE test result, or more specifically a multiple-modification percentage contour map, corresponding to a reaction in which an rhNGF is modified by a polymer of formula A-40K;

FIG. 7 shows a DOE test result, or more specifically a contour line overlay plot, corresponding to a reaction in which an rhNGF is modified by a polymer of formula A-40K;

FIG. 8 shows SDS-PAGE test results as to how a reaction in which an rhNGF is modified by a polymer of formula A is affected by the reaction temperature, with M being molecular-weight-standard protein markers, and lanes 1 and 2 corresponding respectively to modified product LAP2-20K at 5±3° C. and modified product LAP2-20K at 25±2° C.;

FIG. 9 shows SDS-PAGE test results as to how a reaction in which an rhNGF is modified by a polymer of formula A is affected by the reaction time, with M being molecular-weight-standard protein markers, and lanes 1-9 corresponding respectively to the rhNGF, a polymer of formula A-20K, and modified products LAP2-20K whose reaction times are 1 h, 2 h, 4 h, 6 h, 8 h, 16 h, and 24 h respectively; and

FIG. 10 shows curves representing TF-1 cell proliferation stimulated respectively by modified product LAP2-20K and modified product LAP2-40K.

### INFORMATION ON SEQUENCE LISTING

SEQ ID NO. 1: a single-chain rhNGF amino acid sequence

SEQ ID NO. 2: another single-chain rhNGF amino acid sequence

### DETAILED DESCRIPTION OF THE INVENTION

The embodiments described below serve only to illustrate the method and device of the present invention and are not intended to be restrictive of the scope of the invention. The two formula-A polymers used are of “formula A-20K” and “formula A-40-K” respectively, are of different molecular weights, and are reagents provided by Jenkem Technology Co., Ltd. (Beijing), the product codes being Y-PALD-20K and Y-PALD-40K respectively.

Embodiment 1: Formula-A Polymer Reacting with rhNGF to Produce Modified rhNGF

Reaction 1: Preparation of modified product LAP2-20K (1)

A pH 5.5 acetic acid/sodium acetate buffer solution system was added with 5 mL of rhNGF primary liquid of SEQ ID NO. 1 such that the system had a protein content of 0.5 mg/mL.

Sodium cyanoborohydride was then added until a final concentration of 20 mM was reached.

After that, a formula-A polymer of a molecular weight of 20 kD (of formula A-20K) was added such that the molar ratio of the polymer to the rhNGF was 1:1.

The aforesaid reactants were allowed to react at 5±3° C. for 16 h.

The reaction mixture was subjected to an SDS-PAGE test, in which samples were separately stained with barium iodide and Coomassie Brilliant Blue. The test results are shown in FIG. 1.

Reaction 2: Preparation of modified product LAP2-40K (1)

The method was the same as reaction 1, except that a polymer of formula A-40K, which had a molecular weight of 40 kD, was used.

The reaction mixtures of the two reactions were purified by ion exchange chromatography according to the differences between the charges on the unmodified rhNGF and on the modified rhNGF. The resulting modified products are herein named LAP2-20K and LAP2-40K respectively.

As shown in FIG. 1, both modified products LAP2-20K and LAP2-40K were able to be stained with barium iodide and Coomassie Brilliant Blue, and the molecular weights of modified products LAP2-20K and LAP2-40K were greater than that of the rhNGF, greater than the molecular weights of the formula-A-20K polymer and of the formula A-40K polymer respectively, and less than two times the molecular weights of the formula-A-20K polymer and of the formula A-40K polymer respectively (see lanes 2, 3, 4, and 5 in FIG. 1). That is to say, both the formula-A-20K polymer and the formula-A-40K polymer were covalently bound to the rhNGF under the aforesaid conditions, and highly efficient modification of the rhNGF was achieved. Moreover, the modified products were chiefly singly modified.

Reaction 3: Preparation of modified product LAP2-20K (2)

The method was the same as reaction 1, except that the rhNGF used was of the amino acid sequence of SEQ ID NO. 2.

Reaction 4: Preparation of modified product LAP2-40K (2)

The method was the same as reaction 2, except that the rhNGF used was of the amino acid sequence of SEQ ID NO. 2.

The optimal pH values, molar ratios, temperatures, and reaction times of the aforesaid reactions as well as a preferred amount of use of each material/reagent were determined by the following tests.

Embodiment 2: Using the Response Surface Methodology for DOE to Optimize the Reaction Conditions for Modifying rhNGF with Formula-A Polymer

An experiment for investigating the effect of the pH value of the buffer solution and of the molar ratio of the formula-A polymer (of formula A-20K or formula A-40K) to the rhNGF on the modification percentages was designed by the response surface methodology for DOE, with the single-modification percentage and the multiple-modification percentage being the response values. The modification percentages of the samples were measured by SEC-HPLC (size exclusion chromatography-high performance liquid chromatography).

(1) DOE test results corresponding to a reaction in which an rhNGF is modified by a polymer of formula A-20K

The modification percentages of samples corresponding to different conditions were analyzed by SEC-HPLC and are shown in Table 1.

TABLE 1
Modification percentages corresponding to rhNGF modification
by a polymer of formula A-20K, as obtained by SEC-HPLC

| | | | Single- | Multiple- |
|---|---|---|---|---|
| | | Molar ratio | modification | modification |
| Experiment | pH | (Formula | percentage | percentage |
| number | value | A/rhNGF) | (%) | (%) |
| 1 | 5.0 | 1.25:1 | 77.8183 | 7.0448 |
| 2 | 6.0 | 2.0:1 | 81.9687 | 7.5793 |
| 3 | 4.0 | 2.0:1 | 62.2776 | 3.9569 |
| 4 | 5.0 | 1.25:1 | 81.893 | 10.1909 |
| 5 | 5.0 | 0.5:1 | 37.3733 | 1.1276 |
| 6 | 5.0 | 2.0:1 | 85.1351 | 9.5087 |
| 7 | 6.0 | 0.5:1 | 26.6129 | 0.7521 |
| 8 | 4.0 | 1.25:1 | 41.5001 | 0.9821 |
| 9 | 4.0 | 0.5:1 | 19.3581 | 0 |
| 10 | 6.0 | 1.25:1 | 68.8154 | 5.1369 |
| 11 | 5.0 | 1.25:1 | 79.1886 | 7.8777 |

An analysis of variance was performed on the Prob>F value of the single-modification percentage and multiple-modification percentage model, and it was found that the Prob>F value was less than 0.05, indicating that the model was successfully established and significant; in other words, the pH value of the buffer solution and the molar ratio of the formula-A-20K polymer to the rhNGF had a highly significant effect on the modification percentages.

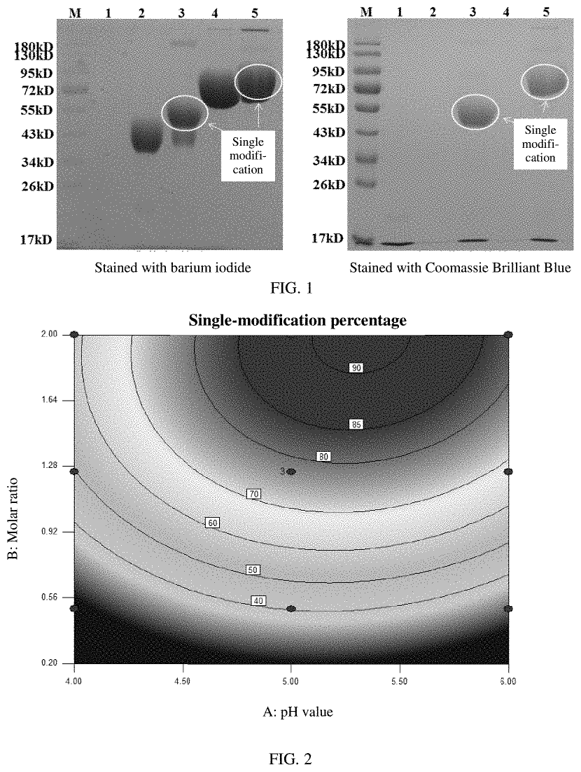

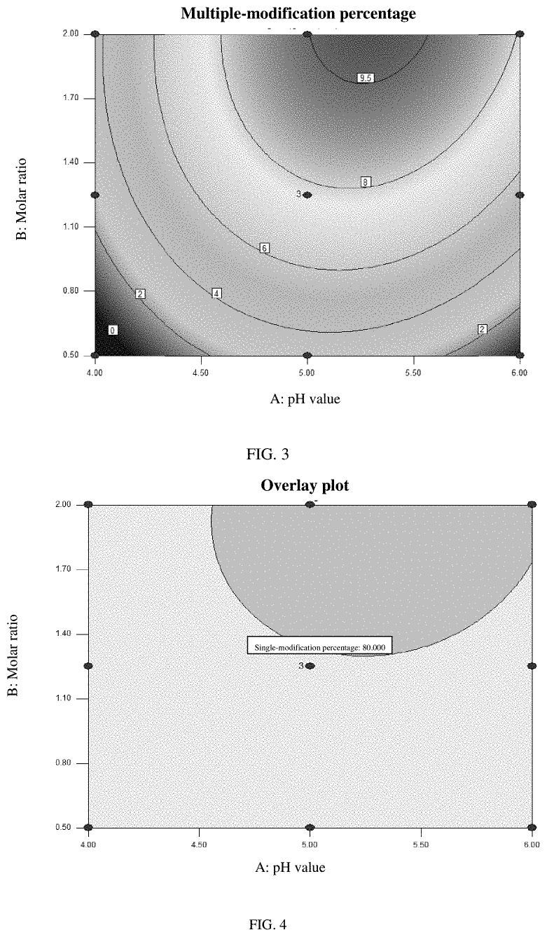

Referring to FIG. 2 and FIG. 3 for the contour line analysis results, the modification percentages increased with the molar ratio and the pH value. Relatively high single-modification percentages were achieved when the molar ratio was greater than 1.3:1 and the pH value ranged from 4.5 to 6.0.

By setting limits to the modification percentages (single-modification percentage ≥80%, multiple-modification percentage ≤15%) and superimposing areas that satisfy those criteria, an overlay plot was obtained as shown in FIG. 4, according to which the modification conditions of the pH value being 5.0-5.5 and the molar ratio being 1.3-1.8:1 were selected.

(2) DOE test results corresponding to a reaction in which an rhNGF is modified by a polymer of formula A-40K

The modification percentages of samples corresponding to different conditions were analyzed by SEC-HPLC and are shown in Table 2.

TABLE 2
Modification percentages corresponding to rhNGF modification
by a polymer of formula A-40K, as obtained by SEC-HPLC

| | | | Single- | Multiple- |
|---|---|---|---|---|
| | | Molar ratio | modification | modification |
| Experiment | pH | (Formula | percentage | percentage |
| number | value | A/rhNGF) | (%) | (%) |
| 1 | 5.0 | 1.25:1 | 74.4577 | 5.4881 |
| 2 | 6.0 | 2.0:1 | 88.3697 | 10.3034 |
| 3 | 4.0 | 2.0:1 | 49.2854 | 1.6031 |
| 4 | 5.0 | 1.25:1 | 65.3191 | 2.9918 |
| 5 | 5.0 | 0.5:1 | 23.129 | 0.6736 |
| 6 | 5.0 | 2.0:1 | 80.3451 | 4.6633 |
| 7 | 6.0 | 0.5:1 | 23.7482 | 0.8591 |
| 8 | 4.0 | 1.25:1 | 31.0797 | 0.8142 |
| 9 | 4.0 | 0.5:1 | 15.9614 | 0 |
| 10 | 6.0 | 1.25:1 | 69.3874 | 4.0761 |
| 11 | 5.0 | 1.25:1 | 68.0486 | 3.8918 |

An analysis of variance was performed on the Prob>F value of the single-modification percentage and multiple-modification percentage model, and it was found that the Prob>F value was less than 0.05, indicating that the model was successfully established and significant; in other words, the pH value of the buffer solution and the molar ratio of the formula-A-40K polymer to the rhNGF had a highly significant effect on the modification percentages.

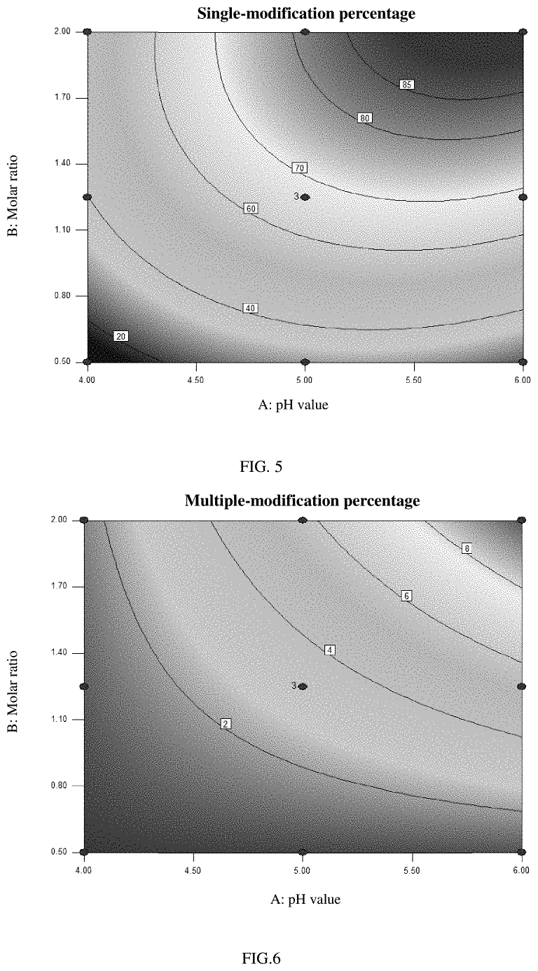

Referring to FIG. 5 and FIG. 6 for the contour line analysis results, the modification percentages increased with the molar ratio and the pH value. Relatively high single-modification percentages were achieved when the molar ratio was greater than 1.7:1 and the pH value ranged from 5.0 to 6.0.

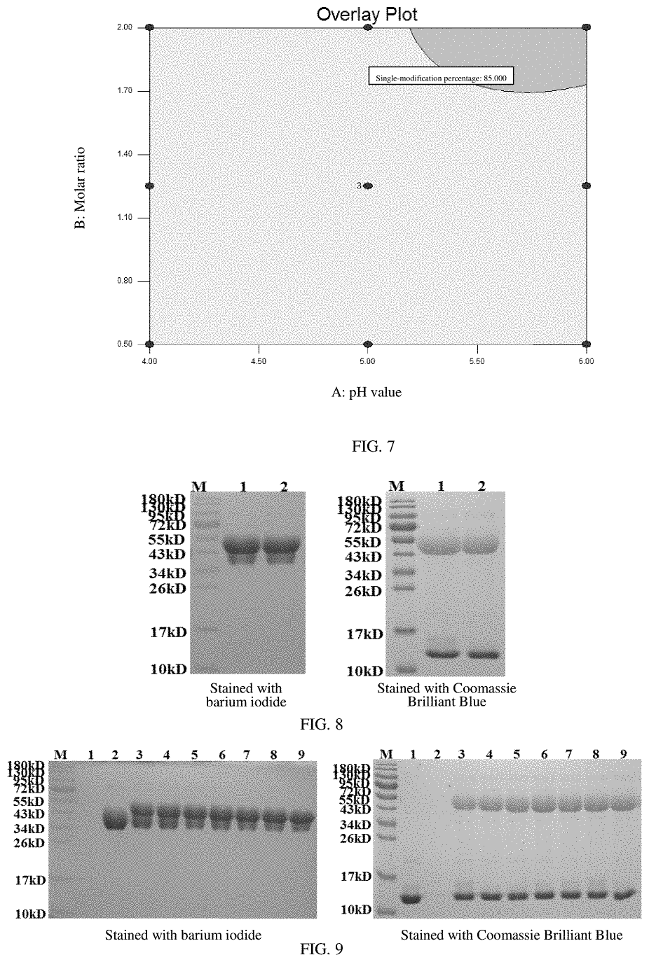

By setting limits to the modification percentages (single-modification percentage ≥85%, multiple-modification percentage ≤10%) and superimposing areas that satisfy those criteria, an overlay plot was obtained as shown in FIG. 7, according to which the modification conditions of the pH value being 5.25-5.75 and the molar ratio being 1.7-2.0:1 were selected.

Embodiment 3: Validation of the Preferred Reaction Conditions Selected from the DOE Test Results for rhNGF Modification

Based on the modification conditions (pH value and molar ratio ranges) of LAP2-20K and LAP2-40K as selected by the response surface methodology for DOE, a test for validating the selected modification conditions of LAP2-20K and LAP2-40K was designed as shown in Table 3.

The modification percentages of the samples were determined by SEC-HPLC, and the results are also shown in Table 3, in which it can be seen that for LAP2-20K, a single-modification percentage >83% and a multiple-modification percentage <13% were achieved with a pH value of 5.0-5.5 and a molar ratio of 1.5-1.8:1, and that for LAP2-40K, a single-modification percentage >85% and a multiple-modification percentage <12% were achieved with a pH value of 5.25-5.75 and a molar ratio of 1.7-2.0:1.

TABLE 3
SEC-HPLC-based validation of the selected modification
conditions of LAP2-20K and LAP2-40K

| | | | Single- | Multiple- |
|---|---|---|---|---|
| | | Molar ratio | modification | modification |
| | pH | (Formula | percentage | percentage |
| Condition | value | A/rhNGF) | (%) | (%) |
| LAP2-20K-a | 5.0 | 1.5:1 | 83.235 | 8.8449 |
| LAP2-20K-b | 5.0 | 1.8:1 | 85.3162 | 10.8887 |
| LAP2-20K-c | 5.25 | 1.65:1 | 87.0759 | 8.6361 |
| LAP2-20K-d | 5.5 | 1.5:1 | 84.8958 | 10.316 |
| LAP2-20K-e | 5.5 | 1.8:1 | 84.3894 | 12.8847 |
| LAP2-40K-a | 5.25 | 1.7:1 | 87.884 | 8.2452 |
| LAP2-40K-b | 5.75 | 2.0:1 | 87.401 | 11.6758 |
| LAP2-40K-c | 5.5 | 1.85:1 | 88.7949 | 10.3086 |
| LAP2-40K-d | 5.25 | 1.7:1 | 86.2399 | 11.4713 |
| LAP2-40K-e | 5.75 | 2.0:1 | 87.5554 | 11.421 |

Embodiment 4: The Effect of Reaction Temperature on rhNGF Modification by a Polymer of Formula A-20K

The method was the same as reaction 1, except that the additional reaction condition of the temperature being 25±2° C. was imposed on reaction 1. The test results are shown in FIG. 8, in which it can be seen that highly efficient rhNGF modification by the formula-A-20K polymer was achieved at 25±2° C. as well as at 5±3° C.

Embodiment 5: The Effect of Reaction Time on rhNGF Modification by a Polymer of Formula A-20K

The method was the same as reaction 1, except that the additional reaction time points (at the end of 1 h, 2 h, 4 h, 6 h, 8 h, and 24 h) were used in reaction 1. The test results are shown in FIG. 9, in which it can be seen that highly efficient rhNGF modification by the formula-A-20K polymer was achieved with a reaction time ≥2 h.

Embodiment 5: Pharmacokinetic Research on Formula-A-Polymer-Modified rhNGF in Rat

1. Purpose of experiment: To compare the half-life of formula-A-polymer-modified rhNGF in rat body with those of unmodified rhNGF and of rhNGF modified by a different polymer

2. Control-group drug and experimental-group drugs:

Unmodified rhNGF LAP1-20K (rhNGF modified by monomethoxy polyglycol propionaldehyde, with a molecular weight of 20 KDa)

Formula-A-polymer-modified rhNGF: LAP2-20K and LAP2-40K

3. Experimental method:

7-to-9-week-old Sprague Dawley (SD) rats were divided into groups of six , each group including three male rats and three female rats. The rats in each group received a single intramuscular injection of the unmodified rhNGF, LAP1-20K, LAP2-20K, or LAP2-40K at 30 μg/kg, respectively.

Blood was collected from the rats before the injection and 5 min, 10 min, 30 min, 1 h, 2 h, 4 h, 6 h, 8.167 h, 12 h, 24 h, and 48 h after the injection, and serum was separated from the collected blood.

Drug concentrations were determined by an enzyme-linked immunosorbent assay (ELISA). Pharmacokinetic parameters were calculated from the drug concentrations and time data by a non-compartment analysis (NCA) in order to analyze the pharmacokinetic characteristics of the unmodified/modified rhNGF in the bodies of the rats.

4. Experimental results: See Table 4.

TABLE 4
Pharmacokinetic parameters of unmodified/modified rhNGF
administered into rat body by single intramuscular injection (X̅ ± S, n = 6)

| | | Unmodified | | | |
|---|---|---|---|---|---|
| | | rhNGF | LAP1-20K | LAP2-20K | LAP2-40K |
| Parameter | Unit | 30 μg/kg | | | |
| AUC₍₀₋ₜ₎ | ng/mL · h | 31.644 ± 9.888 | 1098.575 ± 695.4 | 2174.567 ± 773.015 | 5419.398 ± 1059.728 |
| AUC_{(0-∞)} | ng/mL · h | 31.712 ± 9.964 | 1024.143 ± 372.98 | 2288.911 ± 871.809 | 10133.495 ± 2156.934 |
| MRT₍₀₋ₜ₎ | h | 3.099 ± 0.368 | 15.448 ± 1.576 | 16.219 ± 3.05 | 20.995 ± 1.084 |
| MRT_{(0-∞)} | h | 3.144 ± 0.391 | 17.439 ± 2.765 | 27.593 ± 9.699 | 61.699 ± 29.416 |
| t1/2z | h | 2.674 ± 0.742 | 9.814 ± 2.053 | 19.862 ± 7.276 | 42.858 ± 20.282 |
| Tₘₐₓ | h | 1.063 ± 0.496 | 7.445 ± 1.119 | 9.056 ± 7.555 | 10.806 ± 6.464 |
| Vz/F | mL/kg | 3733 ± 828 | 936.813 ± 584.593 | 197.002 ± 37.886 | 176.248 ± 49.904 |
| CLz/F | mL/h/kg | 1008 ± 234 | 32.23 ± 11.184 | 14.839 ± 5.532 | 3.067 ± 0.634 |
| Cₘₐₓ | ng/mL | 8.210 ± 1.731 | 69.702 ± 25.776 | 89.039 ± 20.553 | 167.613 ± 44.073 |

The experimental results show the following:

1) Half-live

The half-life of the unmodified rhNGF was 2.674 hours.

The half-life of the control-group drug LAP1-20K (rhNGF modified by monomethoxy polyglycol propionaldehyde) was 9.814 hours.

The half-lives of the two formula-A-polymer-modified rhNGF, namely LAP2-20K and LAP2-40K, were respectively 19.862 and 42.858 hours, which are respectively 7.4 and 16 times as long as the half-life of the unmodified rhNGF.

Compared with the half-life of the control-group drug, the half-lives of LAP2-20K and LAP2-40K were both significantly extended (about two- to fivefold).

2) Plasma concentrations

The effective plasma concentrations of the two formula-A-polymer-modified rhNGF molecules showed a more than eightfold increase, and the AUC values an at least thirtyfold increase. Moreover, all the pharmacokinetic parameters of LAP2-20K and LAP2-40K were superior to those of LAP1-20K.

5. Conclusion of the experiment:

The modified rhNGF of the present invention had a higher in vivo plasma concentration and a longer in vivo half-life than the unmodified rhNGF and the rhNGF modified by monomethoxy polyglycol propionaldehyde.

Embodiment 6: TF-1 Cell/MTS Colorimetric Assay of the Bioactivity of Modified rhNGF

1. Purpose of experiment:

To investigate the effect of the rhNGF modification method of the present invention on the bioactivity of an rhNGF

2. Experimental materials and method:

Human erythroleukemia cells (acclimated NGF-dependent TF-1 cells provided by the recombinant protein unit of the National Institutes for Food and Drug Control) in good growth condition were cultured in a basic culture medium (RPMI 1640+10% fetal bovine serum (FBS)) and seeded on a 96-well plate at 5000 cells per well, the volume of each well being 100 μL.

Each well was then added with 100 μL of the to-be-tested unmodified rhNGF, LAP2-20K, or LAP2-40K solution, all of which had been diluted with a basic culture medium and with a gradient dilution factor of 3. The concentrations used were 36, 12, 4, 1.33, 0.44, 0.15, 0.049, and 0.016 nM. Each concentration was applied to two wells.

Once the solution in each well was thoroughly mixed, the mixed solutions were placed into an incubator (37° C., 5% CO₂) for incubation for 72 h.

After that, each well was added with 20 μL of MTS (3-(4,5 -dimethylthiazol-2-yl)-5-(3-carboxymethoxyphenyl)-2-(4-sulfophenyl)-2H-tetrazolium), and the solution in each well was thoroughly mixed and incubated at 37° C. for 3 h.

The optical density (OD) of each well was determined by a plate reader at 492 nm, and the absorbance-concentration curve of each group was generated by the fitting function of software OriginPro 8.

3. Experimental results:

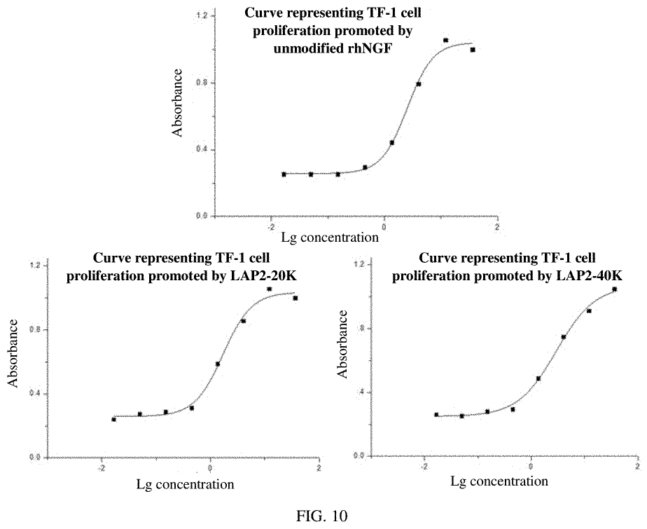

Referring to FIG. 10, the curves representing stimulated TF-1 cell proliferation indicate that both LAP2-20K and LAP2-40K stimulated the proliferation of TF-1 cells. The cell proliferation is in the same dose-dependent manner as unmodified rhNGF.

4. Conclusion of the experiment:

The modified rhNGF provided by the present invention preserved the bioactivity of the unmodified rhNGF in promoting TF-1 cell proliferation experiment, a recognized bioactivity assay of NGF.

1. A modified recombinant human nerve growth factor, obtained from a reaction between a polymer of formula A and a recombinant human nerve growth factor, wherein the polymer of formula A is polymer of N-disubstituted amino acetamino aldehyde derivative and has a weight-average molecular weight of 10 kD-40 kD, 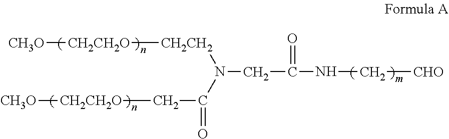
where m is 1 or 2.

2. The modified recombinant human nerve growth factor of claim 1, wherein the recombinant human nerve growth factor is a dimer formed by two identical single-chain of amino acids, and the single-chain of amino acids are selected from the sequences of SEQ ID NO. 1 or SEQ ID NO. 2.

3. The modified recombinant human nerve growth factor of claim 1, wherein the formula A is polymer and the weight-average molecular weight is 20 kD or 40 kD.

4. Use of a polymer of formula A in preparing a nerve growth factor having extended half-life

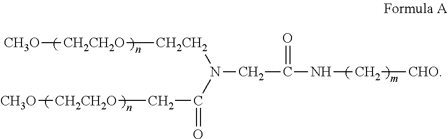

5. A nerve-growth-promoting therapeutic agent, comprising the modified recombinant human nerve growth factor of claim 1 wherein the modified recombinant human nerve growth factor is obtained by covalent binding of the polymer of formula A and an N-terminal α-amino group of the recombinant human nerve growth factor, as following: 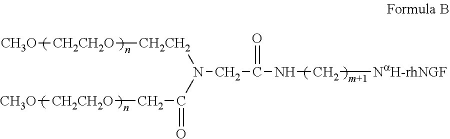
where m is 1 or 2.

6. A therapeutic agent comprising the modified recombinant human nerve growth factor of claim 1.

7. A method for preparing a modified recombinant human nerve growth factor, comprising reacting the polymer of formula A in claim 4 with a recombinant human nerve growth factor.

8. The method of claim 7, wherein the reaction takes place in the presence of sodium cyanoborohydride serving as a reducing agent, the polymer of formula A and the recombinant human nerve growth factor are in a molar ratio of 1-2:1, and the reducing agent, sodium cyanoborohydride, has a final concentration of 20 mM.

9. The method of claim 8, wherein reaction solvent is acetic acid/sodium acetate buffer solution, and a resulting reaction system has a pH value of 5.0-5.8.

10. The method of claim 8, wherein a reaction temperature is 5±3° C. or 25±2° C. and a reaction time is 2 h-24 h.


SEQUENCE LISTING (2 sequences)

SEQ ID NO: 1 - protein, 118 aa, Homo sapiens
SSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVR

SEQ ID NO: 2 - protein, 117 aa, Homo sapiens
SSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAV